(19) United States
(12) Patent Application Publication
(10) Pub. No.: US 2018/0263564 A1
(43) Pub. Date: Sep. 20, 2018

(54) Rehabilitation Footwear Device

(71) Applicant: Francois Avril, Tamarac, FL (US)

(72) Inventor: Francois Avril, Tamarac, FL (US)

(21) Appl. No.: 15/459,144

(22) Filed: Mar. 15, 2017

(51) Int. Cl. A61B 5/00; A43B 3/00; A43C 11/14; A43B 3/12; A61B 5/103

(52) U.S. Cl. CPC A61B 5/6807; A43B 3/0021; A43C 11/1493; A43B 3/128; A43B 3/0036; A43B 3/001; A61B 2562/0247; A61B 5/7282; A61B 5/7405; A61B 5/7445; A61B 5/746; A61B 2505/09; A61B 5/1038

(57) ABSTRACT

A rehabilitation footwear device for assisting in controlling weight application to an injured lower appendage includes a shoe that is configured to position around and reversibly couple to a foot of a user. A control module that comprises a power module and a microprocessor is coupled to the shoe. The microprocessor is operationally coupled to the power module. A plurality of sensors is coupled to and positioned in a sole of the shoe. The sensors are operationally coupled to the microprocessor. The sensors are configured to measure pressure applied to the sole from the foot that is positioned in the shoe and to relay a pressure measurement to the microprocessor. A speaker is coupled to the shoe. The speaker is operationally coupled to the microprocessor. The microprocessor is positioned to compel the speaker to emit an audio alert should the pressure measurement exceed a threshold limit.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to footwear devices and more particularly pertains to a new footwear device for assisting in controlling weight application to an injured lower appendage.

### BRIEF SUMMARY OF THE INVENTION

An embodiment of the disclosure meets the needs presented above by generally comprising a shoe that is configured to position around and reversibly couple to a foot of a user. A control module that comprises a power module and a microprocessor is coupled to the shoe. The microprocessor is operationally coupled to the power module. A plurality of sensors is coupled to and positioned in a sole of the shoe. The sensors are operationally coupled to the microprocessor. The sensors are configured to measure pressure applied to the sole from the foot that is positioned in the shoe and to relay a pressure measurement to the microprocessor. A speaker is coupled to the shoe. The speaker is operationally coupled to the microprocessor. The microprocessor is positioned to compel the speaker to emit an audio alert should the pressure measurement exceed a threshold limit.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a rehabilitation footwear device according to an embodiment of the disclosure.

FIG. 2 is a side view of an embodiment of the disclosure.

FIG. 3 is a top view of an embodiment of the disclosure.

FIG. 4 is a cross-sectional view of an embodiment of the disclosure.

FIG. 5 is a block diagram of an embodiment of the disclosure.

FIG. 6 is an isometric perspective! view of an embodiment of the disclosure.

### DETAILED DESCRIPTION OF THE INVENTION

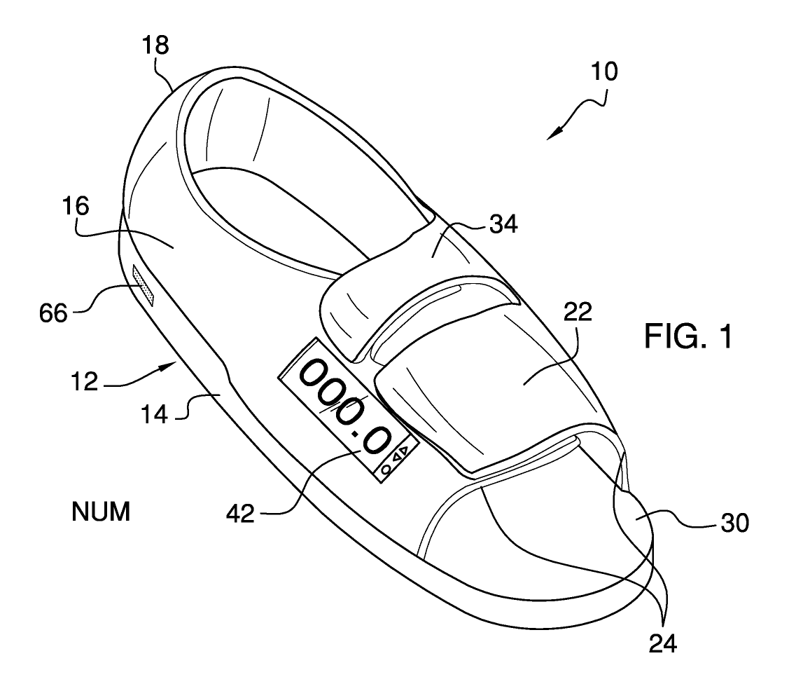

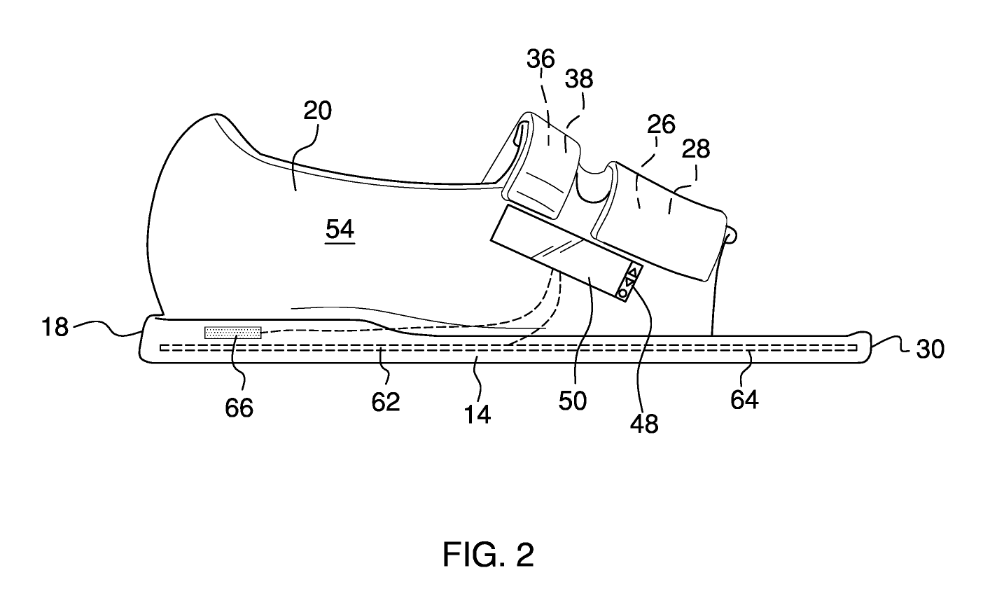

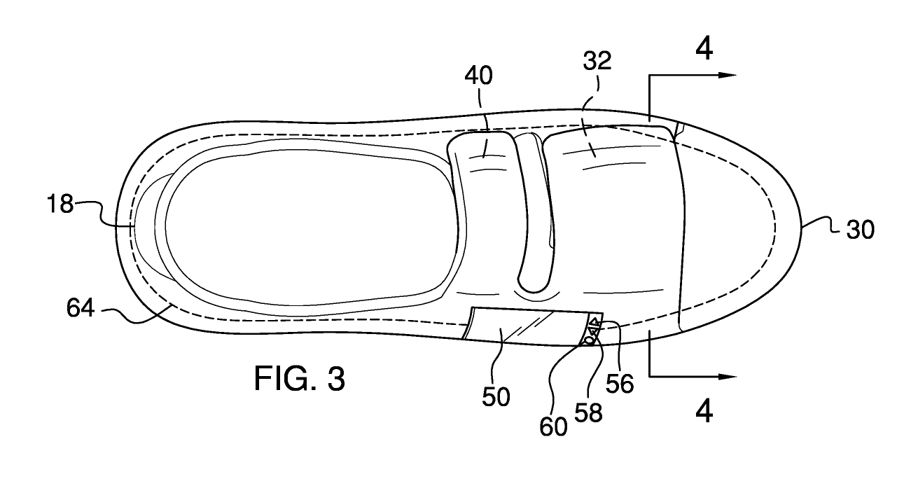

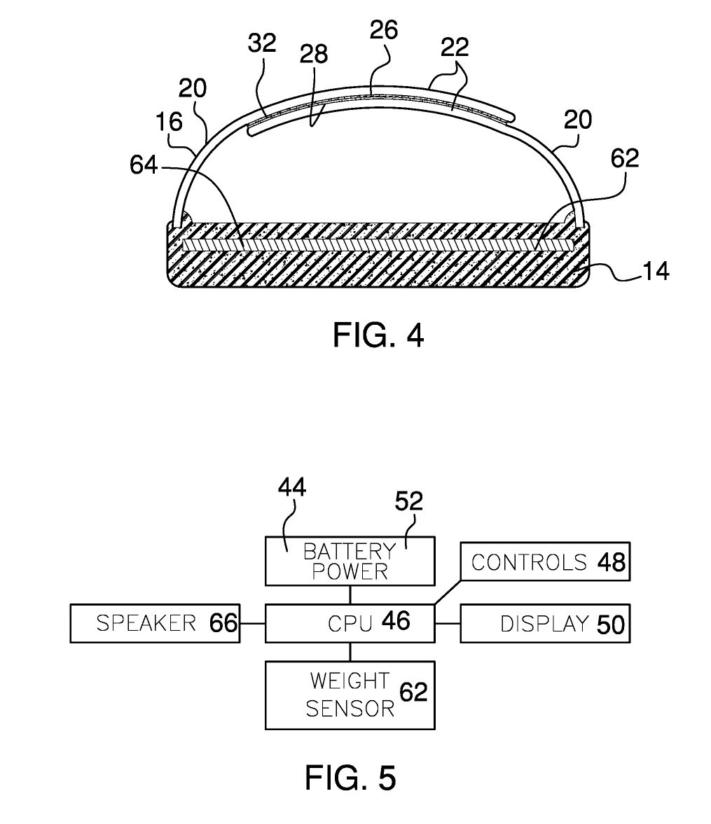

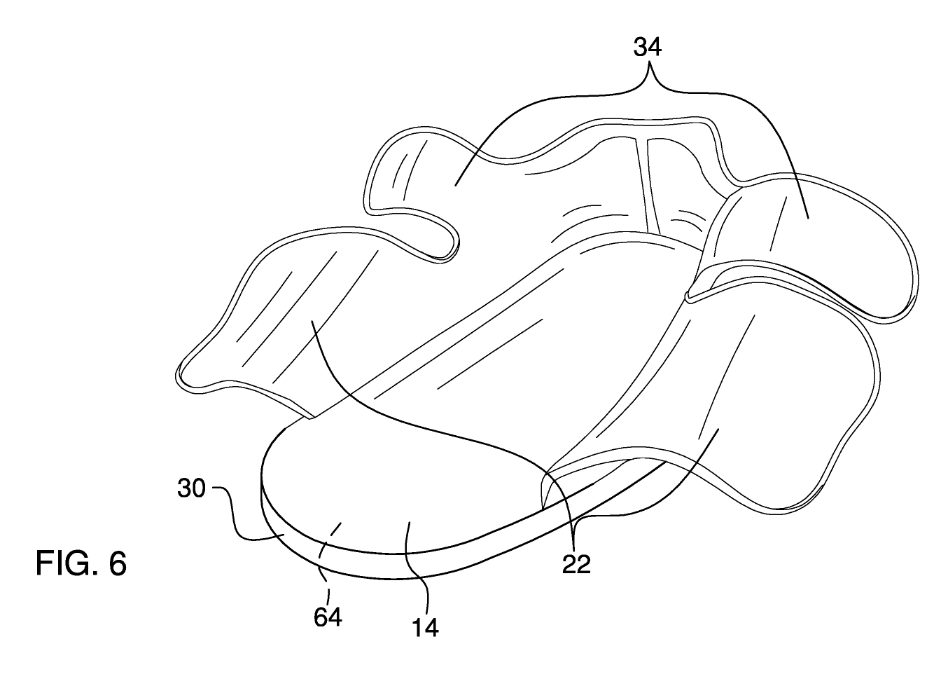

With reference now to the drawings, and in particular to FIGS. 1 through 6 thereof, a new footwear device embodying the principles and concepts of an embodiment of the disclosure and generally designated by the reference numeral 10 will be described.

As best illustrated in FIGS. 1 through 6, the rehabilitation footwear device 10 generally comprises a shoe 12 that is configured to position around a foot of a user. The shoe 12 is configured to reversibly couple to the foot of the user. In one embodiment, the shoe 12 is substantially symmetrically shaped so that the shoe 12 is configured to selectively couple to a left foot and a right foot of the user.

In another embodiment, the shoe 12 comprises a sole 14. A first panel 16 is coupled to and extends from the sole 14 to define a heel end 18 and opposing sides 20 of the shoe 12. Each of a pair of first straps 22 is coupled to and extends from the first panel 16 proximate to a respective opposing end 24 of the first panel 16. A first connector 26 is coupled to a respective first strap 22. A second connector 28 is coupled to a respective first strap 22. The first connector 26 and the second connector 28 are opposingly positioned. The second connector 28 is complementary to the first connector 26. The second connector 28 is positioned to reversibly couple to the first connector 26 to mutually couple the first straps 22 to couple the shoe 12 to the foot of the user. A toe end 30 of the shoe 12 is open. In yet another embodiment, the first connector 26 and the second connector 28 comprise a first hook and loop fastener 32.

In another embodiment, each of a pair of second straps 34 is coupled to and extends from the first panel 16 proximate to a respective first strap 22. A first coupler 36 is coupled to a respective second strap 34. A second coupler 38 is coupled to a respective second strap 34. The first coupler 36 and the second coupler 38 are opposingly positioned. The second coupler 38 is complementary to the first coupler 36. The second coupler 38 is positioned to reversibly couple to the first coupler 36 to mutually couple the second straps 34 to couple the shoe 12 to the foot of the user. In yet another embodiment, the first coupler 36 and the second coupler 38 comprise a second hook and loop fastener 40.

A control module 42 is coupled to the shoe 12. The control module 42 comprises a power module 44, a microprocessor 46, a controller 48, and a screen 50. In one embodiment, the power module 44 comprises at least one battery 52. The microprocessor 46 is operationally coupled to the power module 44. The controller 48 and the screen 50 are operationally coupled to the microprocessor 46. The controller 48 is positioned at a surface 54 of the shoe 12 so that the controller 48 is accessible to the user. The screen 50 is positioned adjacent to the controller 48 so that the screen 50 is configured to be viewed by the user. The controller 48 is positioned to program a threshold limit into the microprocessor 46. The microprocessor 46 is positioned to compel the screen 50 to display the threshold limit that is programmed into the microprocessor 46. The microprocessor 46 also is positioned to compel the screen 50 to flash red as a visual alert to the user should the pressure measurement exceed the threshold limit.

In one embodiment, the controller 48 comprises a first button 56, a second button 58, and a third button 60. The first button 56 is configured to be depressed to compel the microprocessor 46 to increase the threshold limit. The second button 58 is configured to be depressed to compel the microprocessor 46 to decrease the threshold limit. The third button 60 is configured to be depressed to compel the microprocessor 46 to set the threshold limit.

A plurality of sensors 62 is coupled to and positioned in the sole 14 of the shoe 12. The sensors 62 are operationally coupled to the microprocessor 46. The sensors 62 are configured to measure pressure applied to the sole 14 from the foot that is positioned in the shoe 12. The sensors 62 are configured to determine a pressure measurement and to relay the pressure measurement to the microprocessor 46. In one embodiment, the plurality of sensors 62 comprises a second panel 64. The second panel 64 is flexible. In another embodiment, the second panel 64 is shaped substantially complementarily to the sole 14.

A speaker 66 is coupled to the shoe 12. The speaker 66 is operationally coupled to the microprocessor 46. The microprocessor 46 is positioned to compel the speaker 66 to emit an audio alert should the pressure measurement exceed the threshold limit. In one embodiment, the speaker 66 is positioned in the sole 14 proximate to the heel end 18 of the shoe 12.

In use, the second connector 28 is positioned to reversibly couple to the first connector 26 to mutually couple the first straps 22 to couple the shoe 12 to the foot of the user. A toe end 30 of the shoe 12 is open. The controller 48 is positioned to program the threshold limit into the microprocessor 46. The sensors 62 are configured to determine the pressure measurement and to relay the pressure measurement to the microprocessor 46. The microprocessor 46 is positioned to compel the speaker 66 to emit the audio alert should the pressure measurement exceed the threshold limit.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A rehabilitation footwear device comprising:
a shoe configured for positioning around and reversibly coupling to a foot of a user;
a control module coupled to said shoe, said control module comprising a power module and a microprocessor, said microprocessor being operationally coupled to said power module;
a plurality of sensors coupled to and positioned in a sole of said shoe, said sensors being operationally coupled to said microprocessor, said sensors being configured for measuring pressure applied to said sole from the foot positioned in said shoe;
a speaker coupled to said shoe, said speaker being operationally coupled to said microprocessor; and
wherein said sensors are positioned in said sole such that said sensors are configured for determining a pressure measurement and for relaying the pressure measurement to said microprocessor, wherein said speaker is positioned in said shoe such that said microprocessor is positioned for compelling said speaker to emit an audio alert should the pressure measurement exceed a threshold limit.

2. The device of claim 1, further including said shoe being substantially symmetrically shaped such that said shoe is configured for selectively coupling to a left foot and a right foot of the user.

3. The device of claim 1, further including said shoe comprising:
a sole;
a first panel coupled to and extending from said sole defining a heel end and opposing sides of said shoe;
a pair of first straps, each said first strap being coupled to and extending from said first panel proximate to a respective opposing end of said first panel;
a first connector coupled to a respective said first strap;
a second connector coupled to a respective said first strap such that said first connector and said second connector are opposingly positioned, said second connector being complementary to said first connector; and
wherein said second connector is positioned on said respective said first strap such that said second connector is positioned for reversibly coupling to said first connector to mutually couple said first straps for coupling said shoe to the foot of the user such that a toe end of said shoe is open.

4. The device of claim 3, further including said first connector and said second connector comprising a first hook and loop fastener.

5. The device of claim 3, further comprising:
a pair of second straps, each said second strap being coupled to and extending from said first panel proximate to a respective said first strap;
a first coupler coupled to a respective said second strap;
a second coupler coupled to a respective said second strap such that said first coupler and said second coupler are opposingly positioned, said second coupler being complementary to said first coupler; and
wherein said second coupler is positioned on said respective said second strap such that said second coupler is positioned for reversibly coupling to said first coupler to mutually couple said second straps for coupling said shoe to the foot of the user.

6. The device of claim 5, further including said first coupler and said second coupler comprising a second hook and loop fastener.

7. The device of claim 1, further including said power module comprising at least one battery.

8. The device of claim 1, further including a controller positioned at a surface of said shoe such that said controller is accessible to the user, said controller being operationally coupled to said microprocessor, wherein said controller is positioned on said shoe such that said controller is positioned for programming a threshold limit into said microprocessor.

9. The device of claim 1, further including said controller comprising:
a first button configured for depressing to compel said microprocessor to increase the threshold limit;
a second button configured for depressing to compel said microprocessor to decrease the threshold limit; and
a third button for depressing to compel said microprocessor to set the threshold limit.

10. The device of claim 9, further including a screen positioned adjacent to said controller such that said screen is configured for viewing by the user, said screen being operationally coupled to said microprocessor, wherein said screen is positioned on said shoe such that said microprocessor is positioned for compelling said screen to display the threshold limit being programmed into said microprocessor, wherein said microprocessor is positioned for compelling said screen to flash red as a visual alert to the user should the pressure measurement exceed the threshold limit.

11. The device of claim 1, further including said plurality of sensors comprising a second panel, said second panel being flexible, said second panel being shaped substantially complementarily to said sole.

12. The device of claim 1, further including said speaker being positioned in said sole proximate to a heel end of said shoe.

13. A rehabilitation footwear device comprising:
a shoe configured for positioning around and reversibly coupling to a foot of a user, said shoe being substantially symmetrically shaped such that said shoe is configured for selectively coupling to a left foot and a right foot of the user, said shoe comprising:
a sole,
a first panel coupled to and extending from said sole defining a heel end and opposing sides of said shoe,
a pair of first straps, each said first strap being coupled to and extending from said first panel proximate to a respective opposing end of said first panel,
a first connector coupled to a respective said first strap,
a second connector coupled to a respective said first strap such that said first connector and said second connector are opposingly positioned, said second connector being complementary to said first connector, wherein said second connector is positioned on said respective said first strap such that said second connector is positioned for reversibly coupling to said first connector to mutually couple said first straps for coupling said shoe to the foot of the user such that a toe end of said shoe is open, said first connector and said second connector comprising a first hook and loop fastener,
a pair of second straps, each said second strap being coupled to and extending from said first panel proximate to a respective said first strap,
a first coupler coupled to a respective said second strap, and
a second coupler coupled to a respective said second strap such that said first coupler and said second coupler are opposingly positioned, said second coupler being complementary to said first coupler, wherein said second coupler is positioned on said respective said second strap such that said second coupler is positioned for reversibly coupling to said first coupler to mutually couple said second straps for coupling said shoe to the foot of the user, said first coupler and said second coupler comprising a second hook and loop fastener;
a control module coupled to said shoe, said control module comprising:
a power module, said power module comprising at least one battery,
a microprocessor, said microprocessor being operationally coupled to said power module,
a controller positioned at a surface of said shoe such that said controller is accessible to the user, said controller being operationally coupled to said microprocessor, wherein said controller is positioned on said shoe such that said controller is positioned for programming a threshold limit into said microprocessor, said controller comprising:
a first button configured for depressing to compel said microprocessor to increase the threshold limit,
a second button configured for depressing to compel said microprocessor to decrease the threshold limit, and
a third button for depressing to compel said microprocessor to set the threshold limit, and
a screen positioned adjacent to said controller such that said screen is configured for viewing by the user, said screen being operationally coupled to said microprocessor, wherein said screen is positioned on said shoe such that said microprocessor is positioned for compelling said screen to display the threshold limit being programmed into said microprocessor, wherein said microprocessor is positioned for compelling said screen to flash red as a visual alert to the user should the pressure measurement exceed the threshold limit;
a plurality of sensors coupled to and positioned in said sole of said shoe, said sensors being operationally coupled to said microprocessor, said sensors being configured for measuring pressure applied to said sole from the foot positioned in said shoe, wherein said sensors are positioned in said sole such that said sensors are configured for determining a pressure measurement and for relaying the pressure measurement to said microprocessor, said plurality of sensors comprising a second panel, said second panel being flexible, said second panel being shaped substantially complementarily to said sole;
a speaker coupled to said shoe, said speaker being operationally coupled to said microprocessor, wherein said speaker is positioned in said shoe such that said microprocessor is positioned for compelling said speaker to emit an audio alert should the pressure measurement exceed the threshold limit, said speaker being positioned in said sole proximate to said heel end of said shoe; and
wherein said second connector is positioned on said respective said first strap such that said second connector is positioned for reversibly coupling to said first connector to mutually couple said first straps for coupling said shoe to the foot of the user such that a toe end of said shoe is open, wherein said controller is positioned on said shoe such that said controller is positioned for programming the threshold limit into said microprocessor, wherein said sensors are positioned in said sole such that said sensors are configured for determining the pressure measurement and for relaying the pressure measurement to said microprocessor, wherein said speaker is positioned in said sole such that said microprocessor is positioned for compelling said speaker to emit the audio alert should the pressure measurement exceed the threshold limit.
